(19) United States
(12) Patent Application Publication
(10) Pub. No.: US 2007/0196506 A2
(43) Pub. Date: Aug. 23, 2007

(54) IMPROVED TOLERANCE NUTRITIONAL PRODUCT TO SUPPLEMENT HUMAN MILK

(75) Inventors: John Euber, Evansville, IN (US); James Hansen, Evansville, IN (US)

Correspondence Address: NELSON MULLINS RILEY & SCARBOROUGH, LLP, 1320 MAIN STREET, 17TH FLOOR, COLUMBIA, SC, 29201, US

(73) Assignee: Bristol-Myers Squibb Company, Evansville, IN (US)

(21) Appl. No.: 10/358,081

(22) Filed: Feb. 4, 2003

Related U.S. Application Data
(60) Provisional application No. 60/354,240, filed on Feb. 4, 2002.

(51) Int. Cl. A61K 35/20; A61K 31/70

(52) U.S. Cl. 424/535; 514/23

(57) ABSTRACT

The invention relates to a nutritional supplement for human milk in powder or liquid form to feed premature infants comprising protein, fat and carbohydrates wherein the carbohydrate content is limited to no more than 10% dry weight of the nutritional supplement, and the fat content is at least about 35% dry weight, to reduce the increase in osmolality occurring when the supplement is added to human milk and, thus, produce a supplemented human milk that is better tolerated by the infants. Vitamins and minerals can be added.

The present application claims the benefit of U.S. Provisional Application Ser. No. 60/354,240 filed Feb. 4, 2002, which is incorporated herein by reference thereto.

### FIELD OF INVENTION

The present invention relates to low osmolality nutrient supplements for premature infants and methods to support the rapid growth of premature infants by administering nutritionally supplemented human milk to those infants.

### BACKGROUND

Pre-term or premature infants are typically infants born before the 37th week of gestation and/or weighing at birth less than 2500 grams. Many of these infants, because of their developmental immaturity and low weight, present special nutritional needs that normally cannot by met by their mothers' milk or banked human milk. Donor milk, in addition, raises health concerns of potential adulteration with bacteria, viruses or other contaminants.

In general, human milk, because of its nutrient composition and immunological properties, is considered an ideal food for infants. However, human milk is typically too low in proteins and certain minerals such as calcium and phosphorus to meet the needs for rapid growth of many pre-term infants. Protein, a crucial nutrient for infants' growth and synthesis of enzymes and hormones, and certain minerals such as calcium and phosphorus that are needed for appropriate bone development and bone density, must be provided to pre-term infants in the form of human milk nutritional supplements or fortifiers.

Furthermore, the caloric content of human milk typically requires that pre-term infants be fed a volume of milk that is too high to be well tolerated by the infants. Normally, premature infants may tolerate total daily feedings of between 100 to 150 ml per kg of the infants' weight. Since the caloric content of human milk is approximately 67 Kcal per 100 ml of milk (20 Kcal per fluid ounce of milk), and pre-term infants need approximately 120 Kcal per kg of weight per day, human milk can supply only about 80 percent of the infant's energy needs. Thus, to provide a caloric intake that meets the need for pre-term infants to grow rapidly with a volume of milk that the infants can tolerate, the caloric content of the human milk should be supplemented with a source of energy such as carbohydrates, in addition to protein and minerals. For these purposes, nutritional supplements are designed such that, when added to human milk, the supplemented human milk is capable of delivering to the infant approximately 24 Kcal per fluid ounce (approximately 81 Kcal per 100 ml), together with amounts of protein and minerals that are higher than those normally present in human milk.

While the use of nutrient supplements is an attractive solution for the special nutritional needs of pre-term infants, the additional nutrients generally cause an increase in the osmolality of the supplemented human milk over the levels that are typical in human milk. Osmolality refers to the concentration of osmotically-active particles in an aqueous solution per unit weight of solvent, and is expressed in mOsm/kg. When two solutions employing the same solvent but having different osmolality are contacted through a membrane permeable only to the solvent, the solvent will flow from the low osmolality solution to the high osmolality solution. This phenomenon is particularly pronounced when the dissolved compounds are certain species, such as simple carbohydrates and electrolytes, that are known to have high osmotic activity. Other species such as emulsified fats, the form of fat added to nutrient supplements, on the other hand, have low or no osmotic activity. When a hyperosmolar solution, i.e., an aqueous solution having osmolality higher than that of normal body fluids (approximately 300 mOsm/kg of water), is ingested, certain undesirable gastrointestinal side effects may take place. The hyperosmolar solution may cause an osmotic effect in the stomach and small intestine: water is drawn into the gastrointestinal tract to dilute the concentration of the osmotically-active particles. The influx of water into the gastrointestinal tract may cause diarrhea, nausea, cramping, abdominal distension, regurgitation and vomiting.

Carbohydrates are an energy source readily available for incorporation in nutritional supplements. However, they may have high osmotic activity, particularly simple carbohydrates or those carbohydrates that are highly hydrolyzed. Even complex carbohydrates can detrimentally affect the supplemented human milk osmolality since they may be rapidly hydrolyzed by amylase, an enzyme normally present in human milk. As a result, the osmolality of supplemented human milk may be about 90 to 120 mOsm/kg above normal osmolality levels in unsupplemented milk.

### SUMMARY OF THE INVENTION

Briefly, the present invention is directed to a novel nutrient supplement for addition to human milk with a fat content of about 35% by dry weight or more and a carbohydrate content of about 10% by dry weight or less in the nutrient supplement. The present invention is also directed to a novel method for providing supplemental nutrients to a premature infant and to a method of promoting the growth of a premature infant, the methods comprising adding the nutrient supplement of the present invention to human milk and administering the supplemented milk to the premature infant.

Among the several advantages found to be achieved by the present invention, the substitution of fats for carbohydrates in the nutrient supplement results in a smaller increase in the nutritionally supplemented human milk osmolality and, thus, an increased tolerance to supplemented human milk by premature infants.

### DETAILED DESCRIPTION OF THE PREFERRED EMBODIMENTS

In accordance with the present invention, it has been discovered that the substitution of fats for carbohydrates in nutrient supplements that are added to human milk for administration to premature infants results in a supplemented human milk that has an osmolality closer to that of unsupplemented human milk and which is well tolerated by most premature infants. The nutrient supplement of the present invention (when in powder or liquid form) comprises proteins, fats and carbohydrates in various degrees. However, the present invention requires that fat be at least about 35% by dry weight and that the carbohydrate content be limited to no more than about 10% by dry weight. In this manner, osmolality increases resulting from the additional nutrients are less than about 35 to 40 mOsm/kg

Most any fat can be used in the present invention, provided it is suitable for combination with the other components of the supplement. Exemplary fats include soy oil, medium chain triglycerides (MCT oil), corn oil, sunflower oil, safflower oil, coconut oil, palm oil, cottonseed oil, high oleic safflower, high oleic sunflower, and canola oil. The fat source can comprise one or more of these oils. Emulsifiers, such as lecithin, may replace a small portion of the fat composition, but usually not more than 2%.

Any carbohydrates suitable for infant consumption may be used in the present invention. Commercial sources for these carbohydrates are known to the ordinary practitioner of the art. One particular carbohydrate that could be utilized is corn syrup solids.

Protein sources suitable for use in the present invention include most any protein or nitrogen source suitable for infant consumption. These products are commercially available and their commercial sources are known by practitioners of this art. Both, intact and hydrolyzed proteins, such as hydrolyzed whey protein, can be used. Two particular proteins that can be used are low lactose milk protein isolate (Alapro 9405, from NZMP Co.) and hydrolyzed whey protein isolate (BioZate 3, from Davisco Foods).

Vitamins that may be employed include vitamin A, vitamin D, vitamin E, vitamin K1, thiamin, riboflavin, vitamin B6, vitamin B12, niacin, folic acid, panthotenic acid, biotin, and Vitamin C. Mineral nutrients that may be added include calcium, phosphorus, magnesium, zinc manganese, copper, sodium, potassium, chloride, and iron. In the present invention as shown in Table 1, these mineral nutrients were added in the form of salts such as calcium phosphate, calcium glycerophosphate, calcium gluconate, sodium citrate, potassium chloride, potassium citrate, potassium phosphate, magnesium phosphate, ferrous sulfate, zinc sulfate, and cupric sulfate. Other vitamins and minerals that can be added are within the knowledge of the person with ordinary skill in the art who can determine the appropriate amount of vitamins and mineral nutrients following the recommendations of the Committee on Nutrition of the American Academy of Pediatrics or other groups of experts.

The following example describes an embodiment of the invention. Other embodiments within the scope of the claims herein will be apparent to one skilled in the art from consideration of the specification or practice of the invention as disclosed herein. It is intended that the specification, together with the examples, be considered exemplary only. In the example all percentages are given on a dry weight basis unless otherwise indicated.

### EXAMPLE

This Example demonstrates an embodiment of the composition of the nutritional supplement of this invention. Table 1 shows the amount of base nutrients (proteins, fats and carbohydrates), vitamins and mineral nutrients present in 2.84 grams of a nutritional supplement powder. Actual levels of nutrients may be slightly higher to ensure the indicated levels are delivered over shelf life and for all batches of product. The caloric content of those 2.84 grams of powder is approximately 14 Kcal. Thus, this amount of powder is a recommended dose of nutrient supplement to be added to 100 ml of human milk.

TABLE 1
Composition of 2.84 grams of nutritional supplement
powder Caloric content: 14 Kcalories
Caloric Content

| | |
|---|---|
| Energy, kcal | 14.0 |

Powder Base

| | |
|---|---|
| Protein, g | 1.1 |
| Fat, g | 1.0 |
| Carbohydrate, g | 0.23 |
| Ash, g | 0.37 |
| Linoleic Acid, mg | 140 |
| Linolenic Acid, mg | 17 |

Vitamins

| | |
|---|---|
| Vitamin A, IU | 950 |
| Vitamin D, IU | 150 |
| Vitamin E, IU | 4.6 |
| Vitamin K₁, mcg | 4.4 |
| Thiamin, mcg | 150 |
| Riboflavin, mcg | 220 |
| Vitamin B₆, mcg | 115 |
| Vitamin B₁₂, mcg | 0.18 |
| Niacin, mcg | 3000 |
| Folic Acid, mcg | 25 |
| Pantothenic Acid, mcg | 730 |
| Biotin, mcg | 2.7 |
| Vitamin C, mg | 12 |

Minerals

| | |
|---|---|
| Calcium, mg | 90 |
| Phosphorus, mg | 50 |
| Magnesium, mg | 1 |
| Iron, mg | 1.44 |
| Zinc, mg | 0.72 |
| Manganese, mcg | 10 |
| Copper, mcg | 44 |
| Sodium, mg | 16 |
| Potassium, mg | 29 |
| Chloride, mg | 13 |

This embodiment of the invention can be achieved by adding vitamins and minerals to a powder mix to yield a product containing 39.92% proteins, 36.22% fats, and 8.04% carbohydrates, as illustrated in Table 2 that shows an analysis of 100 grams of nutritional supplement powder.

TABLE 2
Analysis per 100 Grams Powder

| | |
|---|---|
| Protein, g | 39.92 |
| Fat, g | 36.22 |
| Carbohydrate (by diff.), g | 8.04 |
| Ash, g | 12.89 |
| Moisture, g | 2.93 |

For the above protein, fat, and carbohydrate composition, the caloric distribution is shown in Table 3. In Table 3, it can be seen that 32.3% of the caloric content of the nutritional supplement has a protein source, 62.6% a fat source, and 5.1% a carbohydrate source.

TABLE 3
Caloric Distribution

| | |
|---|---|
| Protein | 32.3% |
| Fat | 62.6% |
| Carbohydrate (by diff.) | 5.1% |

In this particular embodiment, the protein sources are low lactose milk protein isolate and hydrolyzed whey protein isolate. Table 4 shows the proportion in which these two protein sources are present in 2.84 grams of powder, the amount of powder that is used as a base to describe in Table 1 the composition.

TABLE 4
Protein per 14 Calories (2.84 grams powder)

| | |
|---|---|
| Total Protein, g | 1.1 |
| Whey Protein, g | 0.66 |
| Casein Protein, g | 0.44 |

In this particular embodiment, the fat sources are medium chain triglycerides (MCT oil), soybean oil and lecithin. Table 5 shows the proportion in which these fat sources are present in 2.84 grams of powder.

TABLE 5
Fat per 14 Calories (2.84 grams powder)

| | |
|---|---|
| Total Fat, g | 1.0 |
| MCT Oil, g | 0.70 |
| Soybean Oil, g | 0.30 |
| Lecithin, g | 0.006 |
| Other, g | 0.024 |

In this particular embodiment, the carbohydrate sources are corn syrup solids and lactose. Tables 6 and 6A show alternative embodiments wherein the proportion in which those two carbohydrate sources are present in 2.84 grams of powder in each embodiment.

TABLE 6
Carbohydrate per 14 Calories (2.84 grams powder)

| | |
|---|---|
| Total Carbohydrate, g | 0.228 |
| Corn Syrup Solids, g | 0.043 |
| Lactose, g | 0.005 |
| Others, g | 0.180 |

TABLE 6A
Carbohydrate per 14 Calories (2.84 grams powder)

| | |
|---|---|
| Total Carbohydrate, g | 0.23 |
| Corn Syrup Solids, g | 0.043 |
| Lactose, g | 0.005 |
| Others, g | 0.182 |

Vitamins (vitamin A, vitamin D₃, vitamin E, vitamin K₁, thiamin, riboflavin, vitamin B₆ hydrochloride, vitamin B₁₂, niacinamide, folic acid, calcium pantothenate, biotin, ascorbic acid), and sources of minerals (calcium phosphate, calcium glycerophosphate, calcium gluconate, sodium citrate, potassium chloride, potassium citrate, potassium phosphate, magnesium phosphate, ferrous sulfate, zinc sulfate, cupric sulfate) are added to achieve the human milk fortifier compositions shown as alternative embodiments in Tables 7 and 7A which may be given to infants as a nutritional supplement to human milk.

TABLE 7
Components of 100.0 Kg. of Table 1 Nutrient Supplement
Human Milk Fortifier Base

| | | |
|---|---|---|
| MCT Oil | 24.711 | kg |
| Milk Protein Isolate, Low Lactose | 22.543 | kg |
| (Alapro 9405, from NZMP Co.) | | |
| Hydrolyzed Whey Protein Isolate | 21.992 | kg |
| (BioZate-3, Davisco Foods) | | |
| Soybean Oil | 10.590 | kg |
| Sodium Citrate Dihydrate, Powder | 1.249 | kg |
| Potassium Chloride | 1.015 | kg |
| Potassium Citrate | 0.922 | kg |
| Potassium Phosphate Monobasic | 0.553 | kg |
| Lecithin Concentrate | 0.210 | kg |
| Magnesium Phosphate Dibasic | 0.193 | kg |
| Calcium Phosphate Tribasic, Ultrafine | 4.753 | kg |
| Calcium Glycerophosphate | 4.369 | kg |
| Calcium Gluconate Monohydrate | 3.345 | kg |

Dry Vitamin Premix for Human Milk Fortifier

| | | |
|---|---|---|
| Ascorbic Acid | 746.592 | g |
| Tocopheryl Acetate, DL-Alpha, Dry, 50%, S.D. | 455.070 | g |
| Dry Vitamin A Palmitate Type 250-SD | 187.960 | g |
| Corn Syrup Solids | 182.878 | g |
| Niacinamide | 148.394 | g |
| Calcium Pantothenate | 40.434 | g |
| Vitamin K₁, Dry Phytonadione USP 1% | 18.868 | g |
| Vitamin D₃ Powder | 18.554 | g |
| Biotin, 1% Trituration | 13.361 | g |
| Riboflavin | 10.996 | g |
| Vitamin B₁₂, 0.1% in starch | 8.907 | g |
| Thiamin Hydrochloride | 7.725 | g |
| Pyridoxine Hydrochloride | 6.875 | g |
| Folic Acid | 1.386 | g |

Iron Trituration

| | | |
|---|---|---|
| Corn Syrup Solids | 1045.475 | g |
| Ferrous Sulfate, Heptahydrate | 269.800 | g |
| Ascorbic Acid | 33.725 | g |

Trace Mineral Premix for Human Milk Fortifier

| | | |
|---|---|---|
| Corn Syrup Solids | 349.414 | g |
| Zinc Sulfate, Monohydrate | 98.159 | g |
| Cupric Sulfate, Pentahydrate | 8.427 | g |

TABLE 7A
Components of 100.0 Kg. of Table 1 Nutrient Supplement
Human Milk Fortifier Base

| | | |
|---|---|---|
| MCT Oil | 24.711 | kg |
| Milk Protein Isolate, Low Lactose | 22.543 | kg |
| (Alapro 9405, from NZMP Co.) | | |
| Hydrolyzed Whey Protein Isolate | 21.992 | kg |
| (BioZate-3, Davisco Foods) | | |
| Soybean Oil | 10.590 | kg |
| Sodium Citrate Dihydrate, Powder | 1.489 | kg |
| Potassium Chloride | 1.015 | kg |
| Potassium Citrate | 0.584 | kg |
| Potassium Phosphate Monobasic | 0.553 | kg |
| Lecithin Concentrate | 0.210 | kg |
| Magnesium Phosphate Dibasic | 0.193 | kg |
| Calcium Phosphate Tribasic, Ultrafine | 4.753 | kg |
| Calcium Glycerophosphate | 4.369 | kg |
| Calcium Gluconate Monohydrate | 3.345 | kg |

Dry Vitamin Premix for Human Milk Fortifier

| | | |
|---|---|---|
| Ascorbic Acid | 746.592 | g |
| Tocopheryl Acetate, DL-Alpha, Dry, 50%, S.D. | 455.070 | g |
| Dry Vitamin A Palmitate Type 250-SD | 187.960 | g |
| Corn Syrup Solids | 182.878 | g |
| Niacinamide | 148.394 | g |
| Calcium Pantothenate | 40.434 | g |
| Vitamin K₁, Dry Phytonadione USP 1% | 18.868 | g |
| Vitamin D₃ Powder | 18.554 | g |
| Biotin, 1% Trituration | 13.361 | g |
| Riboflavin | 10.996 | g |
| Vitamin B₁₂, 0.1% in starch | 8.907 | g |
| Thiamin Hydrochloride | 7.725 | g |
| Pyridoxine Hydrochloride | 6.875 | g |
| Folic Acid | 1.386 | g |

Iron Trituration

| | | |
|---|---|---|
| Corn Syrup Solids | 1045.475 | g |
| Ferrous Sulfate, Heptahydrate | 269.800 | g |
| Ascorbic Acid | 33.725 | g |

Trace Mineral Premix for Human Milk Fortifier

| | | |
|---|---|---|
| Corn Syrup Solids | 349.414 | g |
| Zinc Sulfate, Monohydrate | 98.159 | g |
| Cupric Sulfate, Pentahydrate | 8.427 | g |

As various changes could be made in the above methods and compositions without departing from the scope of the invention, it is intended that all matter contained in the above description shall be interpreted as illustrative and not in a limiting sense.

What is claimed is:

1. A human milk supplement comprising;
a. 35% by dry weight or more of a fat component;
b. 10% by dry weight or less of a carbohydrate component; and
c. a protein component.

2. The human milk supplement of claim 1 wherein said fat component is present in said supplement in an amount of at least about 39% by dry weight.

3. The human milk supplement of claim 1 wherein said carbohydrate component is present in said supplement in an amount of no greater than about 8% by dry weight.

4. The human milk supplement of claim 1 wherein said fat component is present in said supplement in an amount of at least about 39% by dry weight and said carbohydrate component is present in said supplement in an amount of no greater than about 8% by dry weight.

5. The human milk supplement of claim 1 wherein said fat component is present in said supplement in an amount of about 40% by dry weight and said carbohydrate component is present in said supplement in an amount of about 8% by dry weight.

6. The human milk supplement of claim 1 wherein said protein component is present in an amount of about 39% by dry weight.

7. The human milk supplement of claim 1 wherein the caloric content of said supplement comprises greater than about 62% fat and less than about 5% carbohydrates.

8. The human milk supplement of claim 1 wherein said fat component comprises medium chain triglycerides.

9. The human milk supplement of claim 1 further comprising protein chosen from the group consisting of whey and casein protein.

10. (canceled)

11. (canceled)

12. The human milk supplement of claim 1 wherein said fat component comprises medium chain triglycerides.

13. A method of feeding a preterm infant comprising administering to said infant a supplement as set forth in claim 1.

14. (canceled)

15. The human milk supplement of claim 1 wherein said supplement is in powder form.

16. The human milk supplement of claim 1 wherein said supplement is in liquid form.

17. The use of (a) a fat component, (b) a carbohydrate component, and (c) a protein component for the preparation of a human milk supplement for promoting the growth of a preterm infant, wherein said fat component is present in said supplement in an amount of 35% by dry weight or more, and said carbohydrate component is present in said supplement in an amount of 10% by dry weight or less, and wherein said supplement is in powder or liquid form.

18. The use of claim 17 wherein said fat component is present in said supplement in an amount of at least 39% by dry weight.

19. The use of claim 17 wherein said carbohydrate component is present in said supplement in an amount of no greater than 8% by dry weight.

20. The use of claim 17 wherein said fat component is present in said supplement in an amount of at least 39% by dry weight and said carbohydrate component is present in said supplement in an amount of no greater than 8% by dry weight.

21. The use of claim 17 wherein said fat component is present in said supplement in an amount of about 40% by dry weight and said carbohydrate component is present in said supplement in an amount of about 8% by dry weight.

22. The use of claim 17 wherein said protein component is present in said supplement in an amount of at least 39% by dry weight.

23. The use of claim 17 wherein the caloric content of said supplement comprises greater than 62% fat and less than 5% carbohydrates.

24. The use of claim 17 wherein said fat component comprises medium chain triglycerides.

25. The use of claim 17 wherein protein component is selected from the group consisting of why protein, casein protein, and combinations thereof.

26. The use of claim 1 wherein said supplement further comprises protein and said fat component comprises medium chain triglycerides.

27. A method of supplementing the nutritional value of human milk comprising adding to said human milk a supplement comprising: (a) 35% by dry weight or more of a fat component, (b) 10% by dry weight or less of a carbohydrate component, and a protein component, wherein said supplement is in a powder or liquid form.

28. The method of claim 27 wherein said fat component is present in said supplement in an amount of at least 39% by dry weight.

29. The method of claim 27 wherein said carbohydrate component is present in said supplement in an amount of about 8% or less by dry weight.

30. The method of claim 27 wherein said fat component is present in said supplement in an amount of at least 39% by dry weight and said carbohydrate component is present in said supplement in an amount of about 8% or less by dry weight.

31. The method of claim 27 wherein said fat component is present in said supplement in an amount of about 40% by dry weight and said carbohydrate component is present in said supplement in an amount of about 8% by dry weight.

32. The method of claim 27 wherein said protein component is present in said supplement in an amount of about 39% by dry weight.

33. The method of claim 29 wherein the caloric content of said supplement comprises greater than 62% fat and less than 5% carbohydrates.

34. The method of claim 27 wherein said fat component comprises medium chain triglycerides.

35. The method of claim 27 wherein said protein component is selected from the group consisting of whey protein, casein protein, and combinations thereof.
